(19) United States
(12) Patent Application Publication
(10) Pub. No.: US 2003/0120149 A1
(43) Pub. Date: Jun. 26, 2003

(54) Fat suppression enhancer for use with magnetic resonance imaging

(76) Inventors: Helen Zinreich Shafer, Owings Mills, MD (US); Eva Zinreich, Owings Mills, MD (US)

Correspondence Address: HAHN LOESER & PARKS, LLP, TWIN OAKS ESTATE, 1225 W. MARKET STREET, AKRON, OH, 44313, US

(21) Appl. No.: 10/041,803

(22) Filed: Jan. 9, 2002

Related U.S. Application Data
(63) Continuation-in-part of application No. 10/036,715, filed on Dec. 21, 2001, now abandoned.

(51) Int. Cl. A61B 5/055

(52) U.S. Cl. 600/421

(57) ABSTRACT

An enhancer for improving the clarity and resolution of magnetic resonance images of anatomical regions having limited or excess fat content, which consists of layer of plasticized polyvinyl chloride having a thickness of 0.5 cm to 4 cm. In use, said layer may take a variety of forms so long as the layer is placed in between the body of the patient being examined and the receiving coils of the magnetic resonance imaging system.

### FIELD OF THE INVENTION

The present invention relates generally to improving the clarity and resolution of images taken during magnetic resonance imaging. More specifically, the present invention relates to a tissue equivalent plastic that improves the clarity and resolution of images taken of anatomical regions which have excess fat content, while also improving the image of non-fatty tissues.

### BACKGROUND OF THE INVENTION

Magnetic resonance imaging, (“MRI”) is a method by which the location, size, and conformation of organs and other structures of the body can be ascertained.

In the typical MRI system, a magnetic field is established across a body to align the spin axes of the nuclei of a particular chemical element, usually hydrogen, with the direction of the magnetic field. The aligned, spinning nuclei execute precessional motions around the aligning direction of the magnetic field. For the aligned, spinning nuclei, the frequency at which they precess around the direction of the magnetic field is a function of the particular nucleus which is involved and the magnetic field strength. The selectivity of this precessional frequency with respect to the strength of the applied magnetic field is very short and this precessional frequency is considered a resonant frequency.

In an ordinary MRI system, after the nuclei have been aligned or polarized, a burst of radio frequency energy at the resonant frequency is radiated at the body of the patient to produce a coherent deflection of the spin alignment of the selected nuclei. When the deflecting radio energy is terminated, the deflected or disturbed spin axes are reoriented or realigned, and in this process radiate a characteristic radio frequency signal which can be detected by an external coil and then analyzed by the MRI system to establish image contrast between different types of tissues in the body.

Two parameters are used to measure the response of the magnetized sample to a disturbance of its magnetic environment. One is T1 or longitudinal relaxation time, the time it takes the sample to become magnetized or polarized after being placed in a external magnetic field; the other is T2, the spin relaxation time, a measure of the time the sample holds a temporary transverse magnetization which is perpendicular to the external magnetic field. Images based on proton density can be modified by these two additional parameters to enhance differences between tissues.

Hydrogen is usually selected as the basis for MRI scanning because of its prominent magnetic qualities. Hydrogen is easily polarized as it has only a single proton nucleus. Further, hydrogen is abundant in water, a major component of the human body. Tissues which have a high content of water, and thus hydrogen and hydrogen protons are deemed “protonated” and provide strong images during MRI. A disadvantage to hydrogen scanning, however, is that water is a major component of most of the tissues and organs of the body and therefore, most of the tissues of the body are imaged by the MRI system, making it difficult to distinguish between the various tissues with similar hydrogen content during MRI scanning.

When scanning for hydrogen atoms, the images formed in magnetic resonance imaging are really a converted visual display of the otherwise invisible radio waves emitted by protons which are detected by the MRI receiving coil. During this process, tissue areas which have no hydrogen atoms emit no radio waves, and thus the MR image of this tissue is black. Tissues which have a high hydrogen content, on the other hand, may emit a large amount of radio waves depending on the scanning criteria. These signals are converted into a correspondingly bright visual display image. Normally grey scale assignment, based upon the relative energy or signal intensities received from the tissues, is utilized in order that the user may more easily distinguish the various tissues and organs imaged. On these grey scale images, low or no signal is designated as black, and very high signals are assigned a lighter shade of grey or white.

Occasionally, abundant tissues which create a bright signal may overwhelm the signal generated by tissues which are less abundant and have different hydrogen, and thus different proton contents. This may visually mask the image produced by the latter tissue and obscure a disease process or anatomy. As an example, breast tissue is fatty tissue and contains high amounts of water and hydrogen atoms, and thus produces a very bright image on the MRI system. The presence of diseased tissue within the breast tissue can be hidden by the bright signal generated by the breast tissue itself. This decreases the effectiveness of MRI for detection and diagnosis of disease within the breast. This masking occurs similarly in other areas of the body containing fatty tissues.

Other areas, such as the orbits of the eye, can also be difficult to image and thus difficult to ascertain different tissues being examined. In this situation, the skin air interface causes unclear or indiscernible images. Furthermore, the neck area can be difficult to image effectively due to almost continuous movement of the neck caused by breathing and/or swallowing by the patient.

Various methods have been used in trying to separate the signals coming from the various tissues of the body and thereby produce more distinct images. One such method involves nullifying the signal received from a certain tissue. This is done by utilizing spin echo and gradient echo presaturation pulse sequences based upon information about subtle differences in the precessional frequency of hydrogen atoms as they associate with fatty versus non-fatty tissues. For example, in order to improve the noticeability of non-fatty tissues which lie in a background of a fatty tissue, the entire tissue is first subjected to a chemically specific saturation radio pulse. This preparatory pulse essentially affects the hydrogen atoms associated with the fat molecules. These pretreated hydrogen atoms have the effect of being briefly deactivated and do not emit a useful signal when the actual imaging portion of the pulse sequence commences. The MR image is then created with little or no signal generated from the fatty tissues. The resultant image will show the non-fatty tissue against a dark background. This process is called chemically selective presaturation of fat, or fat saturation.

This fat saturation process is unreliable however. Because the precessional differences between the fatty and non-fatty tissues are very minute, this technique must be conducted very precisely or non-fatty tissues are inadvertently variably saturated themselves. This problem is further compounded by the fact that the local magnetic environment of tissues changes based upon their position relative to the coil; position in the magnetic bore; and position with respect to organs or tissues with different magnetic susceptibilities such as tissue next to bone, or tissue next to air. Not only is the immediate magnetic environment important, but the actual geometry of the organ or body part plays a major role in determining the fatty tissue's likelihood of being nullified with the fat saturation technique. For example, fat is more likely to be saturated in the rather cylindrical thigh than in the right angle of the ankle.

Further, interpretive problems can arise in several ways. First, if the fat is not saturated effectively, then pathology can be obscured. Second, if the fat is saturated in only portions of the body part being imaged, then the areas not saturated may be misinterpreted as pathologic tissue. Third, drastic alterations in geometry and magnetic susceptibility which naturally occur in the neck, shoulders, ankles and other superficial joints, for example, can lead to inappropriate saturation of non-fatty tissues which are the subject of the examination.

One method occasionally used to improve fat saturation by addressing the above stated limitation of this technique involves placing water bags around the body part being scanned. This technique is useful in that there is improvement in the quality and reliability of the fat saturation technique. This is based on reducing or eliminating the skin-air interface and by effectively changing the perceived geometry of the part of the body being imaged, such as changing the right angle configuration of the ankle to a more favorable cylindrical shape. However, these do not conform to the surface of the body part being imaged.

Further, as water is highly protonated, it creates a correspondingly bright signal surrounding the fatty tissue image. The bright background is a serious disadvantage for this procedure because it is distracting and counteracts the improved visualization produced by using water-filled bags with fat saturation sequences. The bright background is also problematic when adjusting the potentiometers to “window” an image and try to highlight a structure significantly less bright than the adjacent fat. Without the enhancer of the present invention, when “windowing” for these less bright structures, these images will become obscure and lose definition.

Another method that has been developed to improve fat saturation during magnetic resonance imaging is the placing of a fat saturation material such as a fluorocarbon on or around the body part of the patient being examined, eliminating the skin air interface and reducing anatomical imaging problems. This is accomplished by placing a bag containing the fat saturation enhancing material around the body part to be imaged. However, disadvantages such as the necessity to provide bags of different sizes and shapes in order to be placed around the body part to be imaged is inherent in this method.

Thus, there is a need to provide a product for clarifying images of areas of the anatomy which contain either limited or excess amounts of fat during magnetic resonance imaging, which will better conform to the surface contours of the area being imaged.

### SUMMARY OF THE INVENTION

In view of the foregoing, it is an object of the present invention to provide an enhancer for improving images of anatomical areas being imaged by MRI.

It is another object of the present invention to provide an enhancer for improving the image of anatomical areas containing excess fat cells adjacent or within the tissue or anatomy of the area being imaged by MRI.

It is a further object of the present invention to provide an enhancer for clarifying images of areas with excess fat content, that allows for imaging of all parts of the body without pre-shaping the product to conform to the part of the body being imaged by MRI.

It is still another object of the present invention to provide an enhancer for clarifying images of areas with excess fat content that is easily used by radiologists and radiology technicians during MRI imaging.

It is yet another object of the present invention to provide an enhancer for clarifying images of areas with excess fat content being imaged by MRI that is relatively inexpensive to produce.

In order to achieve these objects, an enhancer consisting of a material that has a specific gravity within the range of the specific gravity of the tissue of the body being imaged is utilized. This tissue equivalent plastic is placed between the receiving coil of the magnetic resonance imaging system and the patient being examined. The enhancer is effective so long as it is placed between the body of the patient being examined and the receiving coils of the MRI system. In one embodiment, the enhancer is placed upon the area of the patient's body surrounding the tissue to be imaged. In another embodiment, the enhancer of the present invention is positioned at any location between the receiving coil of the MRI system and the body of the patient being examined. The enhancer is manufactured from plasticized or highly plasticized polyvinyl chloride having a thickness in the range of 0.5 to 4 cm.

These and other objects of the present invention will become more readily apparent from a reading of the detailed description taken in conjunction with the drawings.

### BRIEF DESCRIPTION OF THE DRAWINGS

FIG. 1 is a perspective view of a patient in a magnetic resonance imaging system, illustrating the enhancer of the present invention in the form of a sheet placed upon an area of the body to be imaged.

FIG. 2A is a frontal view of the enhancer of the present invention formed as a mask to fit around the orbits of the eyes of the patient for imaging the same.

FIG. 2B is a side view of the enhancer of the present invention as in FIG. 2A, having an optional strap for retaining the enhancer on the face of the patient being examined.

FIG. 3 is a cross sectional view of the supporting surface of a magnetic resonance imaging system having the enhancer of the present invention placed thereon.

FIG. 4 is a cross sectional view of the enhancer of the present invention shaped as a form for positioning and retaining the patient during magnetic resonance imaging.

### DETAILED DESCRIPTION OF THE PREFERRED EMBODIMENT

Reference will now be made in detail to the present preferred embodiments of the present invention, examples of which are illustrated in the accompanying drawings. Whenever possible, the same reference numbers will be used throughout the drawings to refer to the same or like parts.

As fatty tissue is highly protonated, this tissue produces a strong signal when scanned and appears as bright, or often white on the magnetic resonance image. This bright signal can mask less abundant, non-fatty tissues that are within or surrounded by fatty tissues. Many times, this non-fatty tissue is the area being focused upon when examining these tissues during an MRI examination. Because of the bright signal being emanated by the surrounding fatty tissues, the signal being produced by the non-fatty tissue may be obscured by the fatty tissue signal.

It is therefore beneficial to suppress the image produced by the fatty tissues in order to better examine the non-fatty tissues that are the focus of the examination. It is especially necessary to suppress the fatty tissue images when examining breast tissue, or tissues in or around the orbits of the eyes of the patient, and tissues within the neck of a patient.

Areas with a limited amount of fat content are also difficult to image effectively if surrounded by fat because of an overshadowing effect which is difficult to “window” with width and level potentiometers.

The present invention acts as an enhancer to facilitate removal of images generated by fatty tissues during MRI. This enhancer is composed of plasticized polyvinyl chloride (“PVC”) having a thickness in the range of 0.5 cm to 4 cm. Other materials having similar properties, including a specific gravity of approximately 1.0, while also being pliable may also be utilized. An example of a composition from which the enhancer of the present invention may be manufactured is produced by Dennis Chemical Company under the product name Clear Hot Melt (soft), product number PX-7514-D. The Dennis Chemical Company product no. PX-7514-D is a plasticized polyvinyl chloride containing dispersion grade polyvinyl chloride resin (30-40%), plasticizer for liquid polyvinyl chloride dispersion (30-40%), and zinc and zinc compound heat stabilizer. The enhancer may also include tint dispersant, containing two pigmented dispersion color additives: ultramarine blue and R/S phthalocyanine blue. The enhancer material can be used in different manners in order to achieve the desired result of clarification of the images of the anatomy having excess fat content. It is important that the material is flexible in order to conform to the surface of the area being imaged to reduce the skin-air interface. Rigid materials are not effective, unless they are molded to conform to the contours of the anatomical area being imaged, such as the neck or shoulders, for example. The enhancer material can be placed upon the body of the patient, on or around the area of the tissue or anatomy to be imaged, like a second skin of the patient. This decreases the skin-air interface.

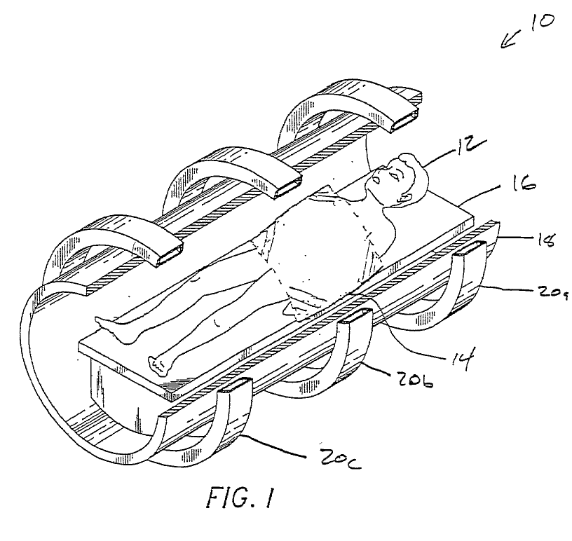

As can be seen in FIG. 1, there is shown a patient 12 positioned upon a supporting surface 16 of a MRI system shown generally at 10. System 10 is such as is readily known in the pertinent art of radiology and imaging. Placed upon the patient is a sheet 14 of the enhancer of the present invention, so as to enhance the clarity and resolution of the images produced while imaging the chest area of the patient. The sheet is composed of plasticized polyvinyl chloride, or material with similar properties, and can be formed into countless different shapes or sizes to allow for the imaging of various portions of the body.

The fat suppression enhancer of the present invention is particularly useful during imaging of the neck, orbits of the eye, and breast areas of human patients. Due to problems with susceptibility artifact and chemical shift artifact, fat suppression in the neck area of a patient is problematic. The usual chemical shift frequency selective fat suppression applied during MRI scans fails at regions below the mandible. Various products have been developed which are said to improve fat saturation throughout the neck on T2 W scans and post gadolinium enhanced fat suppressed T1 W scans. The present invention improves the ability of frequency selective fat suppression to decrease the signal intensity of the fat located in the neck region.

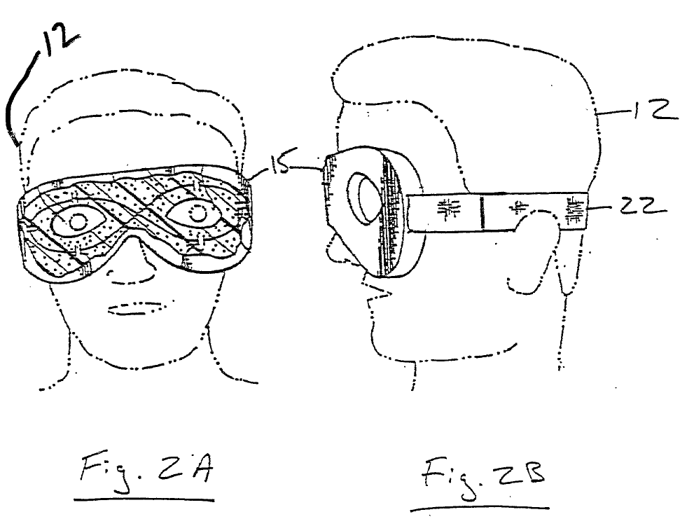

As is shown in FIGS. 2A and 2B, the enhancer of the present invention is formed as a mask 15 to surround the orbits of the eye of the patient being examined. Orbits of eyes have high fat content and therefore present the same problems as set forth above. Although the plasticized enhancer material of the present invention has sufficient adhesive properties, as shown in FIG. 2B, the eye mask 15 may optionally have a strap 22 attached to the mask 15 to aid in retaining the mask 15 in the proper position for imaging the orbits of the eyes of the patient 12.

Placing the enhancer of the present invention on or around the portion of the body desired to be imaged also decreases the skin air interface and thus further increases the clarity of the image produced by the MRI system. The magnetization of the body of the patient differs from that of the air layer surrounding the body. The magnitude of the magnetization is greatly changed in the boundary area between the body and the air layer. Therefore, the static field is disturbed in the area surrounding this boundary area. Placing the enhancer of the present invention between the body of the patient and the receiving coil stabilizes the static magnetic field surrounding the body of the patient.

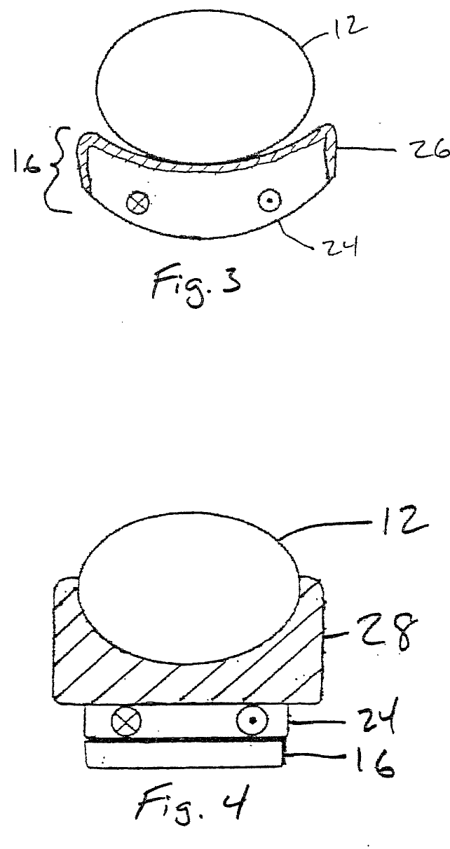

As shown in FIG. 3, a further embodiment of the present invention is illustrated where there is the cross section of the body of the patient 12 positioned upon the supporting surface 16 having a receiving coil 24 placed therein. On the upper and lateral surfaces of the supporting surface 16, there is a layer of sheet 26 of the enhancing material of the present invention. Layer 26 may be permanently affixed upon the upper surface of the supporting surface 16, or may be placed below a cover to reduce wear and damage from cleaning of layer 26. Alternatively, layer 26 may be temporarily placed on the upper surface of the supporting surface between the patient 12 and the receiving coil 24.

Yet another embodiment of the present invention is shown in FIG. 4. In this embodiment the enhancer of the present invention may be shaped into a form 28 for positioning and restraining movement of the patient 12 during imaging. The form 28 is affixed upon the supporting surface and the patient is positioned within form 28 prior to imaging.

It is to be understood that a layer of the enhancer material of the present invention may be configured in a variety of ways so long as the enhancer is positioned between the body of the patient 12 and the receiving coil 24 system 10. Further, the thickness of the enhancer material varies from patient to patient, and is different for each part of the anatomy being imaged.

Although the principles, preferred embodiments and preferred operation of the present invention have been described in detail herein, this is not to be construed as being limited to the particular illustrative forms disclosed. They will thus become apparent to those skilled in the art that various modifications of the preferred embodiments herein can be made without departing from the spirit or scope of the invention as defined by the appended claims.

1. An apparatus for suppressing images of fat cells during magnetic resonance imaging comprised of a layer of plasticized polyvinyl chloride between 0.5 cm and 4 cm thick.

2. An apparatus as recited in claim 1, wherein said layer is placed upon the upper surface of a supporting surface of said magnetic resonance imaging system being used to image a biological body.

3. An apparatus as recited in claim 1, wherein said layer comprises a sheet of said plasticized polyvinyl chloride which is temporarily placed upon a biological body during magnetic resonance imaging.

4. A method of suppressing the image created by fat cells during magnetic resonance imaging wherein a layer of plasticized polyvinyl chloride having a thickness of 0.5 cm to 4 cm is placed between a biological body being imaged and the receiving coils of the magnetic resonance imaging system.

5. A method as recited in claim 4, wherein said layer is placed upon the upper surface of a supporting surface of a magnetic resonance imaging system being used to image a biological body.

6. A method as recited in claim 4, wherein said layer comprises a sheet of said plasticized polyvinyl chloride which is temporarily placed upon a biological body during magnetic resonance imaging.

7. An apparatus for improving the clarity and resolution of anatomical images taken during magnetic resonance imaging comprised of a layer of plasticized polyvinyl chloride between 0.5 cm and 4 cm thick.

8. An apparatus as recited in claim 7, wherein said layer is placed upon the upper surface of a supporting surface of said magnetic resonance imaging system being used to image a biological body.

9. An apparatus as recited in claim 7, wherein said layer comprises a sheet of said plasticized polyvinyl chloride which is temporarily placed upon a biological body during magnetic resonance imaging.
